(19) United States
(12) Patent Application Publication
(10) Pub. No.: US 2013/0210085 A1
(43) Pub. Date: Aug. 15, 2013

(54) Methods and Systems for Pretreatment of Biomass Solids

(71) Applicants: PETER H. KILNER, VISALIA, CA (US); THOMAS P. GRIFFIN, VISALIA, CA (US); BERNARD COOKER, VISALIA, CA (US); ROGER WEINBERG, WILMINGTON, NC (US)

(72) Inventors: PETER H. KILNER, VISALIA, CA (US); THOMAS P. GRIFFIN, VISALIA, CA (US); BERNARD COOKER, VISALIA, CA (US); ROGER WEINBERG, WILMINGTON, NC (US)

(73) Assignee: EDENIQ, VISALIA, CA (US)

(21) Appl. No.: 13/745,403

(22) Filed: Jan. 18, 2013

Related U.S. Application Data
(60) Provisional application No. 61/587,719, filed on Jan. 18, 2012.

(51) Int. Cl. C12P 19/14

(52) U.S. Cl. CPC C12P 19/14
USPC 435/99

(57) ABSTRACT

A method for the pretreatment of biomass solids includes hydrating the biomass solids to form a biomass slurry, shear treating the biomass solids, and hydrolyzing the biomass solids in the presence of reactive enzymes in a pressure hydrolysis zone. Shear treatment of the biomass solids reduces the particle size of the biomass solids, modifies the particle or slurry morphology, and/or ruptures the cell walls of the biomass solids. The pressure hydrolysis zone includes a high-shear, high-pressure, low-temperature heat exchange and reaction zone and a low-pressure, low-temperature polishing zone. Sugars formed from the biomass solids treated in accordance with the methods described above may be used to produce various biofuels.

### RELATED APPLICATION

The present application claims the benefit of U.S. provisional patent application Ser. No. 61/587,719, filed Jan. 18, 2012, the disclosure of which is incorporated by reference herein in its entirety.

### FIELD OF THE INVENTION

The present application relates to the field of biofuel production, and more specifically to methods and systems for pretreating biomass solids for further use in biofuel production processes.

### BACKGROUND OF THE INVENTION

Many U.S. based companies have developed low-cost technology for the conversion of sugar to non-ethanol hydrocarbon fuels, including Amyris (diesel and jet fuel), LS9 (gasoline, diesel, and jet fuel), Virent (gasoline), and Menon & Associates (gasoline, diesel, and jet fuel). Without a source of low-cost sugars here at home, such companies turn to other countries for producing sugar-based fuels, exporting technology overseas and, with it, jobs and fuel production. With world sugar prices at or near all-time highs, this competition for sugar will only exacerbate sugar shortages and inflame the food vs. fuel debate. The hundreds of millions of tons per year of agricultural residues such as corn stover, wheat straw, and wood residues in the U.S.—coupled with the 500 million tons per year of energy crops which could be grown on the 60 million acres of marginal land in the U.S.—could be converted to over 50 billion gallons per year of renewable liquid transportation fuels if cellulosic sugars could be produced here in the U.S. at costs below that of conventional sugar production in foreign countries. This would have a positive impact on the U.S. economy by providing jobs and lessening U.S. dependence on foreign oil.

Many companies have been developing processes to convert cellulosic biomass to sugar intermediates. Conventional processes have failed to achieve compelling production economics primarily due to three factors: 1) use of acids and bases in the pretreatment process, which elevates costs by necessitating expensive materials of construction, by producing problematic byproducts with associated disposal issues, and by forming impurities which undermine the downstream conversion processes; 2) high loadings of expensive cellulosic enzymes; and 3) long saccharification reaction times (several days), resulting in low throughput capacity.

For the production of cellulosic ethanol, enzyme secreting yeasts have been developed that enable the combination of saccharification and fermentation, an option that is not easily incorporated in the micro-organisms capable of converting sugars to hydrocarbon fuels. In addition, a rotor stator colloid mill has been used in conjunction with conventional, multi-day saccharification to produce cellulosic sugars.

### SUMMARY OF THE INVENTION

The terms “invention,” “the invention,” “this invention” and “the present invention” used in this patent are intended to refer broadly to all of the subject matter of this patent and the patent claims below. Statements containing these terms should not be understood to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to the entire specification of this patent, all drawings and each claim.

In an embodiment, a method for the pretreatment of biomass solids includes hydrating the biomass solids to form a biomass slurry, shear treating the biomass solids, and hydrolyzing the biomass solids in the presence of reactive enzymes in a pressure hydrolysis zone. Shear treatment of the biomass solids reduces the particle size of the biomass solids, modifies the particle or slurry morphology, and/or ruptures the cell walls of the biomass solids. The pressure hydrolysis zone includes a high-shear, high-pressure, low-temperature heat exchange and reaction zone and a low-pressure, low-temperature polishing zone.

In some embodiments, the heat exchange and reaction zone includes a plug flow reactor that provides for radial mixing and intentionally limited back mixing of the biomass solids in the biomass slurry to provide sustained contact between the biomass solids and the reactive enzymes and facilitate conversion of the biomass solids into sugar-rich intermediates.

In other embodiments, the polishing zone includes a continuous stirred tank reactor that provides additional residence time to further facilitate conversion of the biomass solids into sugar-rich intermediates.

In certain embodiments, the plug flow reactor operates at a pressure of from about 1,000 psi to about 10,000 psi and a temperature of from about 25° C. to about 140° C. In further embodiments, the plug flow reactor operates at a pressure of from about 1,000 psi to about 5,000 psi and a temperature of from about 35° C. to about 100° C. In yet other embodiments, the plug flow reactor operates at a pressure of from about 1,000 psi to about 2,500 psi and a temperature of from about 35° C. to about 50° C.

In some embodiments, the continuous stirred tank reactor operates at an operating temperature of less than about 70° C. and at a pressure corresponding to the saturation pressure of the biomass slurry at the operating temperature.

In other embodiments the step of hydrating the biomass solids includes a continuous process comprising adding coarsely ground biomass solids into a water stream in a disperser to form the biomass slurry and then passing the biomass slurry into a heat exchanger. In embodiments, the heat exchanger may have an operating temperature of from about 120° C. to about 250° C. and the pressure of the heat exchanger corresponds to the saturation pressure of the biomass slurry at the operating temperature. In certain embodiments, the operating temperature of the heat exchanger is about 180° C. and the pressure of the heat exchanger corresponds to the saturation pressure of the biomass slurry at the operating temperature.

In further embodiments, doping chemicals may be added to the biomass slurry for pH adjustment or control, and/or one or more enzymes may be added to the biomass slurry to help initiate or accelerate downstream enzymatic hydrolysis reactions.

In some embodiments, the biomass slurry is at least about 13 weight percent solids. In other embodiments, the biomass slurry is at least about 20 weight percent solids. In further embodiments, the biomass slurry is at least about 30 weight percent solids.

In yet further embodiments, the step of shear treating the biomass solids includes passing the biomass slurry through at least two particle size reduction mills (including but not limited to, one or both being rotor stator colloid mills) arranged in a series configuration. In other embodiments, the particle size reduction mills reduce a substantial amount of the biomass solids to a particle size of less than about 100 microns. In other embodiments, the particle size reduction mills reduce a substantial amount of the biomass solids to a particle size of less than about 50 microns. In yet other embodiments, the particle size reduction mills reduce a substantial amount of the biomass solids to a particle size of less than about 30 microns.

In certain embodiments, the modification of particle morphology arises from frictional, impact, centrifugal or cavitational forces and results in the cellular liberation of saccharides or saccharide precursors.

In other embodiments, the operating temperature of the biomass slurry in the particle size reduction mills is from about 120° C. to about 250° C., and the pressure of the biomass slurry corresponds to the saturation pressure of the biomass slurry at the operating temperature.

The methods described herein result in a sugar-rich aqueous solution suitable for subsequent chemical, biochemical or enzymatic conversion to valuable fuels, chemicals, or solvents. The sugar-rich aqueous solution may be suitable for synthesis of gasoline-like, jet fuel-like, or diesel-like surrogates, additives, or alternatives.

### BRIEF DESCRIPTION OF THE DRAWINGS

Illustrative embodiments of the present invention are described in detail below with reference to the following drawing figures:

FIG. 1 is a diagram of an exemplary system for pretreatment of biomass solids according to one embodiment of the invention.

FIG. 2 is a graph showing exemplary test results of biomass solids treated according to an embodiment of the invention.

FIG. 3 is a is a graph showing exemplary test results of biomass solids treated according to an embodiment of the invention.

FIG. 4 is a is a graph showing exemplary test results of biomass solids treated according to an embodiment of the invention.

FIG. 5 is a is a graph showing exemplary test results of biomass solids treated according to an embodiment of the invention.

FIG. 6 is a is a graph showing exemplary test results of biomass solids treated according to an embodiment of the invention.

FIG. 7 is a is a graph showing exemplary test results of biomass solids treated according to an embodiment of the invention.

### DETAILED DESCRIPTION OF EMBODIMENTS OF THE INVENTION

The subject matter of embodiments of the present invention is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

Certain embodiments of the invention incorporate process improvements to existing rotor stator colloid mill technology to enable the integration of feed pretreatment and saccharification reactions. The modifications involve substantially improving the contacting efficiency between enzymes and substrate by means of improved equipment design, increasing the localized pressure of the dispersion environment in a series of stages, and adding cellulosic enzymes into totally enclosed, pressurized, controlled reaction zones. Embodiments of the invention result in improvements in process simplicity, throughput rates, conversion levels, and product quality. An exemplary rotor stator colloid mill is described in U.S. patent application Ser. No. 12/547,830, filed Aug. 26, 2009 and published as U.S. 2010/0055741 on Mar. 4, 2010, the entire contents of which are incorporated by this reference.

The present application applies integrated pretreatment and saccharification of any biomass feedstock to produce cellulosic sugars at costs comparable to sugarcane based sugars, with low impurities, to enable economic production of liquid transportation fuels.

In certain embodiments, feedstock for a method of the invention will include corn stover. The corn stover may be provided in baled or pellet form. Other feedstock, including but not limited to agricultural residues, switchgrass, sorghum, sugar cane bagasse, wood feedstocks and wood-derived byproducts (e.g., pulp) may be incorporated into methods described herein.

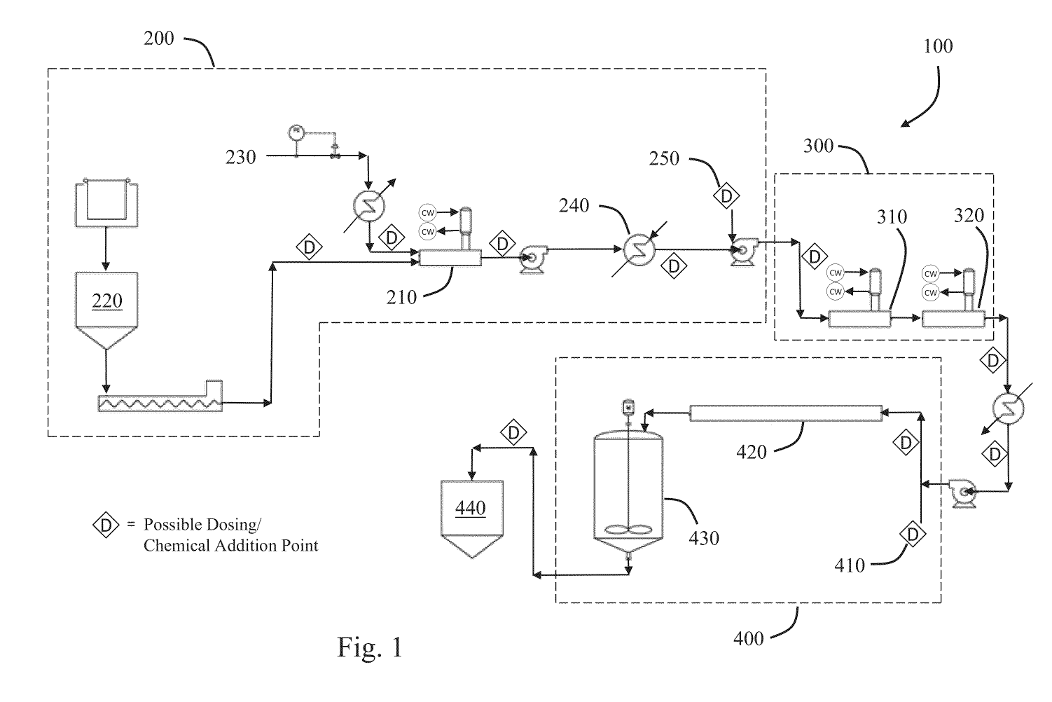

A diagram of an exemplary system for pretreatment of biomass solids is illustrated in FIG. 1. The system (100) according to FIG. 1 includes the general zones described in further detail below. These zones include a biomass dispersion and hydration zone (200), a particle morphology management zone (300), and a pressure hydrolysis and enzyme introduction zone (400).

Biomass Dispersion and Hydration Zone (200)

Known methods for dispersion and hydration include feeding ground biomass and water to an agitated mixing tank, and then applying elevated temperatures and pressures to the tank for several hours to fully hydrate the biomass. High temperature hydration before milling has been shown to decrease hydrolysis time and help sterilize the biomass (i.e., kill microorganisms). This conventional approach, however, has proven difficult to apply to slurry concentrations above 20 wt %.

In some embodiments, in the biomass dispersion and hydration zone (200) pre-processed biomass solids are introduced to a disperser (210) by a gravity hopper (220). Characteristic particle sizes for these solids are typically less than 2 mm, though other starting particle sizes may be utilized as applicable. A water supply (230) is also introduced as a separate inlet stream, and the particles are dispersed and slurried at (or near) room temperature in the disperser (210).

This continuous slurry flow may then be immediately directed toward a heat exchanger/reactor (“hydrator”) (240). In certain embodiments, the hydrator (240) is a single tube pass through a tube-tube heat exchanger, attaining temperatures up to 250° C. and at elevated pressures corresponding to the saturated steam condition. For example, the slurry can be heated to a temperature of from about 120° C. to about 250° C., or more particularly to a temperature of about 180° C., with pressures corresponding to the saturation pressure of the slurry at the given temperature. It will be recognized, however, that other heat exchanger types, temperatures and pressures may be provided. The hydrator (240) allows for both thorough particle wetting (initially via physical association) and also for some initial breakdown of cellulose into sugar precursors/oligomers via the addition of waters of hydration and the associated decrease in cellulose polymer chain length (i.e., molecular weight).

Additives such as doping chemicals for pH adjustment and control may also be, but do not have to be, provided (250) in this zone as desired. Exemplary suitable doping chemicals include, but are not limited to, ionic liquids such as ammonium hydroxide, sodium hydroxide, magnesium hydroxide, calcium hydroxide, sulfuric acid, nitric acid, phosphoric acid, aqueous solutions of each of these, and mixtures thereof. Pressurized gases and/or liquids, including but not limited to oxygen, peroxides, and/or other oxidation agents may also be provided in order to accelerate desired oxidation reaction mechanisms, for example, lignin degradation.

The result of the biomass dispersion and hydration zone (200) is a heated slurry of coarsely-ground (“first grind”) biomass solids, attaining high solids loading. In certain embodiments, the slurry contains greater than about 13 wt. % solids. In yet other embodiments, the slurry contains greater than about 20 wt. %, or greater than about 30 wt. % solids. From the biomass dispersion and hydration zone (200) the heated slurry is then directed to the particle morphology management zone (300).

Attributes of the biomass dispersion and hydration zone (200) include: in-line mixing of pre-processed solids and water; close-coupled mixing and hydration; the ability to quickly and reliably raise and hold at desired hydration temperature; the potential for enhanced internal agitation and turbulence (for example, by way of an internal impact plate in the hydrator (240) to prevent re-agglomeration); and continuous operations and high effective solids loadings.

Particle Morphology Management Zone (300)

Known methods for pretreating biomass particles involve the use of a single, multi-stage particle morphology management mill to mechanically pre-treat the biomass. One such multi-stage particle morphology management mill is a rotor stator colloid mill, including but not limited to a mill such as that available from Edeniq, Inc. under the trade name Cellunator™. This process is limited by the incoming dry ground particle size from the hydration vessel. Thus, the minimum allowable gap size must be increased, reducing the overall milling effectiveness of the particle morphology management mill. “Morphology management,” as this term is used herein, refers to one or more of the following mechanisms: particle size reduction, rupture, compression, de-agglomeration, shape deformation, slurry homogenization and aspect ratio modification.

Embodiments of the present invention may include passing the slurry coming from the biomass dispersion and hydration zone (200) into two distinct three-stage particle morphology management mills (310, 320) in series. The particle morphology management mills (310, 320) may include a shear cutting mechanism. In certain embodiments, each of these particle morphology management mills (310, 320) is a rotor stator colloid mill, for example, as described above. Each particle morphology management mill (310, 320) is designed to progressively liberate cellular contents through aggressive particle management shear techniques. The first particle morphology management mill (310) can rupture, compress, and deagglomerate larger particles, especially those with high aspect ratio, while the second particle morphology management mill (320) includes minimum rotor/stator gap settings to maximize overall particle cell liberation. It is anticipated that this configuration will minimize wear on the particle morphology management mills (310, 320). The particle shear zones may operate at elevated temperatures (including but not limited to from about 120° C. to about 250° C.) and at pressures corresponding to the saturation pressure of the biomass slurry at the given temperature.

The particle morphology management zone (300) thus provides biomass particle size reduction and aspect ratio modification, as well as cellular shear forces that expose a high quantity of the available saccharides and saccharide precursors. In some embodiments, a substantial amount of the biomass particles are reduced to a particle size of less than about 100 microns. In other embodiments, a substantial amount of the biomass particles are reduced to a particle size of less than about 50 microns. In yet other embodiments, a substantial amount of the biomass particles are reduced to a particle size of less than about 30 microns. Smaller particle sizes reduces particle mass and volume, increasing surface area available for enzymatic conversion reactions, and thus significantly reducing the hydrolysis/saccharification reaction times required the pressure hydrolysis and enzyme introduction zone (400).

In further embodiments, approximately 90% of the biomass particles are reduced to a particle size of less than about 100 microns. In certain embodiments, approximately 90% of the biomass particles are reduced to a particle size of less than about 50 microns. In yet further embodiments, approximately 90% of the biomass particles are reduced to a particle size of less than about 30 microns.

In some embodiments, approximately 95% of the biomass particles are reduced to a particle size of less than about 100 microns. In other embodiments, approximately 95% of the biomass particles are reduced to a particle size of less than about 50 microns. In yet other embodiments, approximately 95% of the biomass particles are reduced to a particle size of less than about 30 microns.

In some embodiments, there may be a target particle size that is optimum, and the optimum may be different than reducing the particles to “smallest possible size.” Performance versus cost considerations, for example, may result in selection of a larger particle size. In addition, very fine particles may have a deleterious impact on downstream (separations) operations.

Attributes of the particle morphology management zone (300) include: shear mechanism cutting of the biomass solids; the possibility of combining multiple size reduction mechanisms; the use of multiple, close-coupled particle morphology management mills (310, 320) in series; the use of multiple stages in each particle morphology management mill (310, 320) sequenced by gap size; continuous flow through the particle morphology management mills (310, 320) supporting continuous flow through the entire system (100); high initial solids loading (as described above); high temperature and saturated pressure operations; and the use of centrifugal forces for morphology control. In addition to particle size reduction, the particle morphology management zone may also facilitate rupture of the cell walls of the biomass solids, as well as rapid swelling and compressing of ruptured cells, which will further aid in conversion of the biomass solids into sugars.

Pressure Hydrolysis and Enzyme Introduction Zone (400)

Known saccharification processes involve feeding the biomass slurry post-particle mill management to a stirred tank reactor for saccharification at ambient pressures. It has been determined that, while as many as 50% of the primary particles from current particle size reduction processes may be below 50 microns in diameter, these small particles tend to re-agglomerate in the saccharification reactor. This can reduce the effective surface area and limit otherwise immediate access of enzymes to the cellulose and hemicellulose fibers.

In accordance with the present invention, it has been found that enzymatic hydrolysis can be accelerated by conducting the saccharification process at high pressures followed by dosing enzymes in a turbulent zone. Such methods will enable the enzymes to attack the primary particles before they can agglomerate. With reference to FIG. 1, this process occurs in the pressure hydrolysis and enzyme introduction zone (400).

In this zone, slurried and milled solids from the particle morphology management zone (300) may be immediately subjected to an additional high-shear treatment to ensure that primary particles from the particle morphology management zone (300) do not re-agglomerate, which would decrease available surface area (negating some of the benefit of the particle morphology management zone (300)). The high-shear treatment may be provided via a range of fluid mechanics management devices or mechanisms, e.g., processing through one or more diameter transitions (i.e., orifice plate(s)) provided by impact shear, by way of impingement on an internal plate element or bluff body, etc.

The fine slurried particles (many of which will have particle sizes as described above) may then be fed directly to a very high-pressure, low-temperature, small volume shell-tube exchange reactor, which also provides residence time (minutes) at these conditions for hydrolysis/saccharification. Exemplary process conditions in the exchange reactor include pressures of around 500 psi or greater, temperatures of around 70° C. or less, and a pipe size of around 4″ diameter or less. In other embodiments, the pressure in the exchange reactor can be from about 1,000 to about 10,000 psi, or even from about 1,000 to about 5,000 psi or about 1,500 to about 2,500 psi. In yet other embodiments, the temperature in the exchange reactor can be from about 25° C. to about 140° C., or even from about 35° C. to about 100° C. or 35° C. to about 50° C.

Similarly, the pipe diameter can vary depending on flowrate vs. desired pipe residence time, enzyme injection strategy, and the use and functionality of the downstream continuous stirred tank reactor section, and in some embodiments can vary from about 4″ to about 6″. It will be recognized that pipe sizes can be scaled to system capacity according to known principles.

Reactive enzymes may also be introduced into the pressure hydrolysis and enzyme introduction zone (400) as needed for lignocellulose conversion to sugars (e.g., ligninases, cellulases, and hemicellulases). Exemplary enzyme packages may be provided by any suitable provider, including, but not limited to, Novozyme, Genencor, DSM, and Edeniq.

Other chemicals and/or additives may also be, but do not have to be, introduced in this zone (410) as desired for pH adjustment and control. Suitable chemicals and/or additives include ionic liquids such as ammonium hydroxide, sodium hydroxide, magnesium hydroxide, calcium hydroxide and combinations thereof. In addition, saccharification enzymes and/or gases such as oxygen or inert gases saturated with reaction catalysts may be dosed at this same location to further facilitate the lignocellulose conversion to sugars. The pressure hydrolysis and enzyme introduction zone (400) provides intimate and sustained contact between the enzymes (and optional other additives) and the small-particle, slurried solid substrate, and also affords its full residence time (on the order of, e.g., a few minutes or a few tens of minutes) for the desired conversion of lignocellulosic-derived components to useful sugar-rich intermediates. The heat exchanger/reactor by which this process occurs may be designed as a plug flow reactor (420), which provides considerable radial mixing but limited axial back-mixing—conditions that further facilitate high integral conversion of the desired reactions.

A continuous stirred tank reactor (430) may be provided downstream of the plug flow reactor (420), in series, to afford additional residence time at similar temperatures (but reduced, saturation pressure) for further reaction conversion to be achieved. The net product is a slurry containing the cellulosic sugars which can be filtered to remove solids prior to being deployed in complementary downstream processes for conversion into fuel. This slurry may be stored in a storage tank (440) until ready for use.

Attributes of the pressure hydrolysis and enzyme introduction zone (400) according to the present invention include: very high pressures (1,500-10,000 psi); reactants are confined to very low volume/residence time; the plug flow reactor (420) operates in the high pressure zone and the continuous stirred tank reactor (430) provides “polishing” residence time; the zone operates continuously; saccharification occurs at a high conversion rate and in a short residence time; and the zone allows for high effective solids loadings relative to current operations, and in particular as compared to batch-fed operations.

It is noted that while FIG. 1 depicts various locations where chemicals and/or saccharification enzymes may be added to the system (indicated by the diamond-shaped D's in the figure), it will be recognized that these dosing/addition points are by no means limiting. Other chemical addition points could be provided in the system.

Sugars formed from the biomass solids treated in accordance with the methods described above may be used to produce various biofuels, including but not limited to ethanol, butanol, other oxygenated gasoline additives, synthetic gasoline, biodiesel and aviation fuel, as well as synthetic replacements for petrochemical products.

The invention is further illustrated by the following examples, which are not to be construed in any way as imposing limitations upon the scope thereof.

### EXAMPLES

Description of Experimental Unit:

The purpose of the experimental equipment was to determine the effect of orifi of different internal diameters on the particle size distribution of pumped biocellulosic slurry and to measure the sugars which are released from the solid matrix in consequence of this pretreatment. The equipment was operated by charging wet particulate biocellulosic material and deionized water into an agitated conically-based feed tank, from whence it was transferred by gravity to a Moyno progressive cavity pump (PCP) and thence to a Hydra Cell slurry diaphragm pump (HCP). The latter was rated for 3 gpm at 3,000 psig discharge pressure. The slurry was heated to the required process temperature in a Tube Heat Exchanger, which was heated by regulated low pressure steam. Process temperatures were measured after the HCP and after the Heat Exchanger. Pressures were similarly measured.

The slurry flowed through a Orifice Canister, which holds zero to four orifi, normally manufactured from ruby and lodged in a 316 SS holder, which was screwed into place. The unit was started up on water and switched to the slurry feed once the flows were established. Samples of the feed and of the slurry after the Orifice Canister and immediately before the Feed Tank were taken every 20 to 30 minutes. The samples were analyzed for particle size distribution using a laser dispersion based instrument, the solids of the samples were measured via microwave-heated dryer with weighing capability and the samples were also saccharified.

The saccharification was conducted in the laboratory, in duplicate, in shaken 100 ml glass flasks. The slurry was run at 10 wt % in the flasks, with 20% by weight Trio enzyme solution relative to the glucan. The samples were prepared by the doping with mineral acid or base to adjust the pH to 5.0, followed by the addition of 100 microliters of Alpen antibiotic per 100 gm of mixture and the same concentration of Lactrol antibiotic.

The liquid phase of the saccharifications was analyzed by HPLC for glucose, xylose, cellobiose and arabinose at t=0, 2, 6, 24 and 48 hours. Knowing the solids of the samples and the cellulose and hemicellulose contents of the substrate employed, the sugars released through saccharification were expressed relative to the total potential sugar release at 100% yield. The sugars were dominated by glucose and xylose, in the approximate ratio of 2/1, with traces of arabinose. No other sugars were observed.

Discussion of Data:

The samples from each orifice recirculation run were saccharified, as described above and the relative sugar yield for each sample one was plotted as a function of saccharification time. Orifice IDs, solids levels, raw material sources, flow rate ranges and process temperatures are shown in each figure. The sugars accumulate rapidly in the first 6 hours in the flask and the rate then rapidly falls by 24 hours, with the terminal sample at 48 hour being close to the maximum sugar release at infinite time.

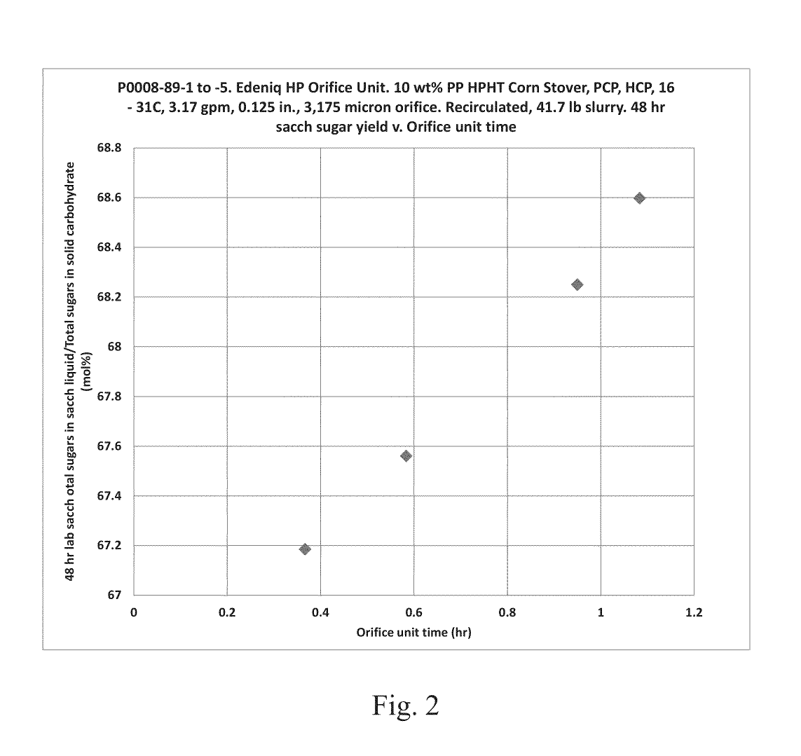

FIG. 2 illustrates the result of plotting the 48 hour saccharin yields for a given series of slurry samples from one run in the orifice unit. The orifice unit was operated for 1.1 hours and during this time the lab saccharin sugar yield at 48 hours improved from 67.2% to 68.6%.

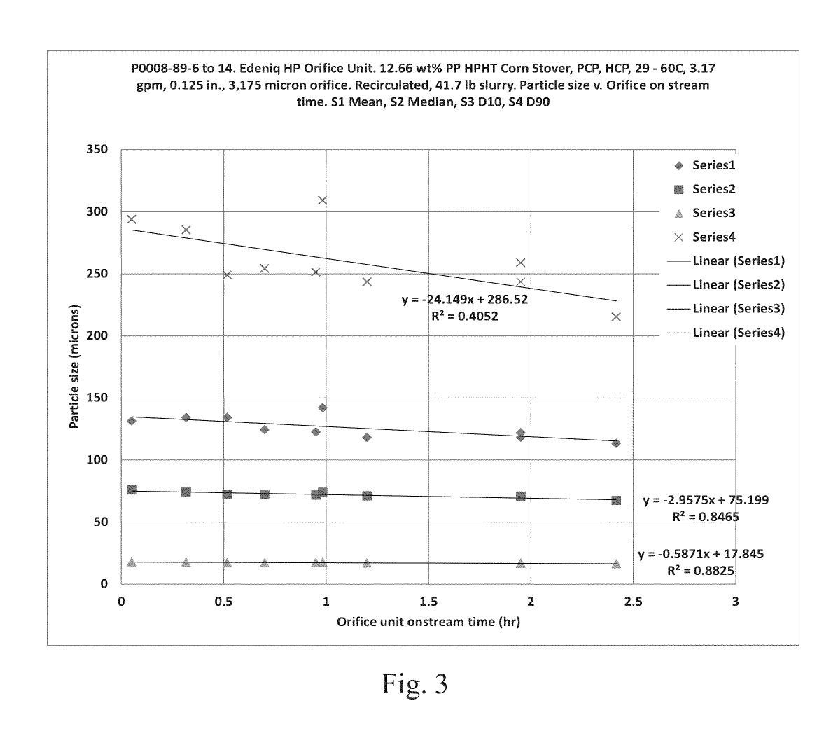

FIG. 3 illustrates the slurry particle size distributions of the samples from run P0008-89-6 to -14. Here, the mean, median, D10 and D90 particle sizes are plotted as functions of the time on stream in the recirculated orifice unit. All indices of particle size show ongoing decline in FIG. 3, as the material is recirculated through the orifice unit.

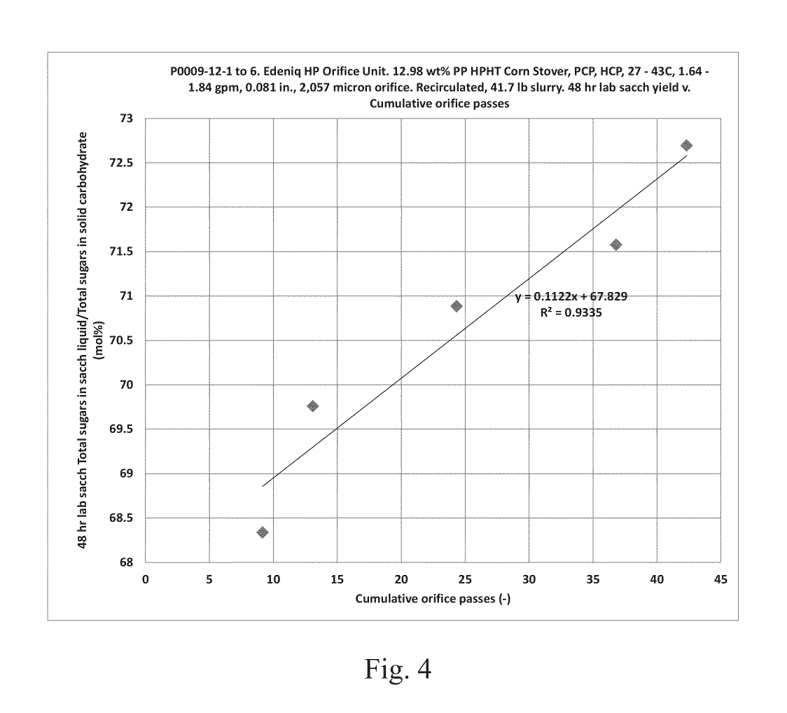

FIG. 4 shows the result of plotting the 48 hour saccharin yields for a given series of slurry samples from one run in the orifice unit (P0009-12-1). The orifice unit was operated for over 40 passes and during this time the lab saccharin sugar yield at 48 hours improved from about 68% to 73%.

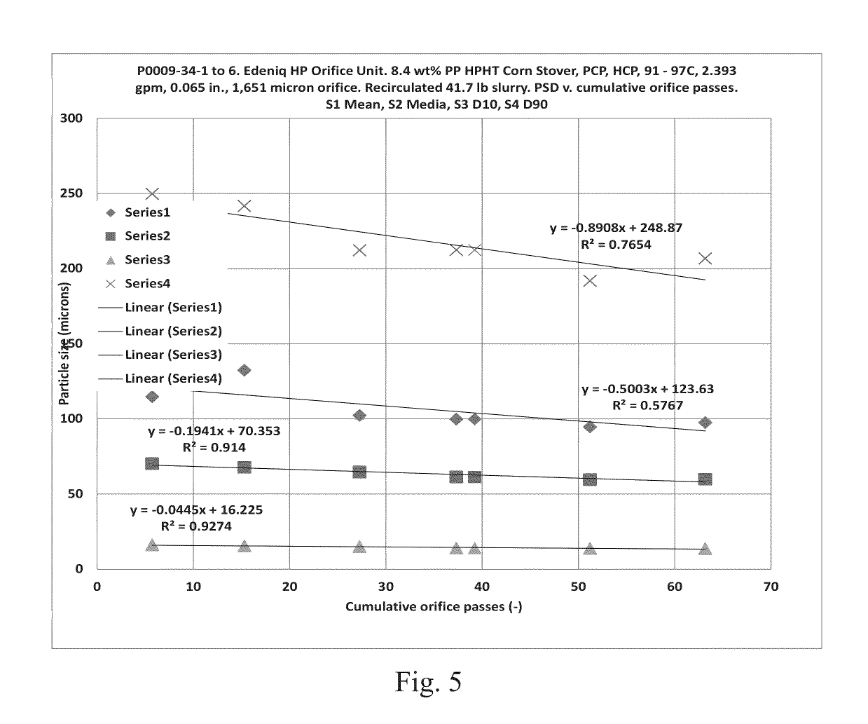

FIG. 5 illustrates the decay of the particle size with ongoing processing for a later run, P0009-34, but the horizontal axis is the cumulative number of orifice passes by the recirculated flow. Again, all indices of particle size fall with processing.

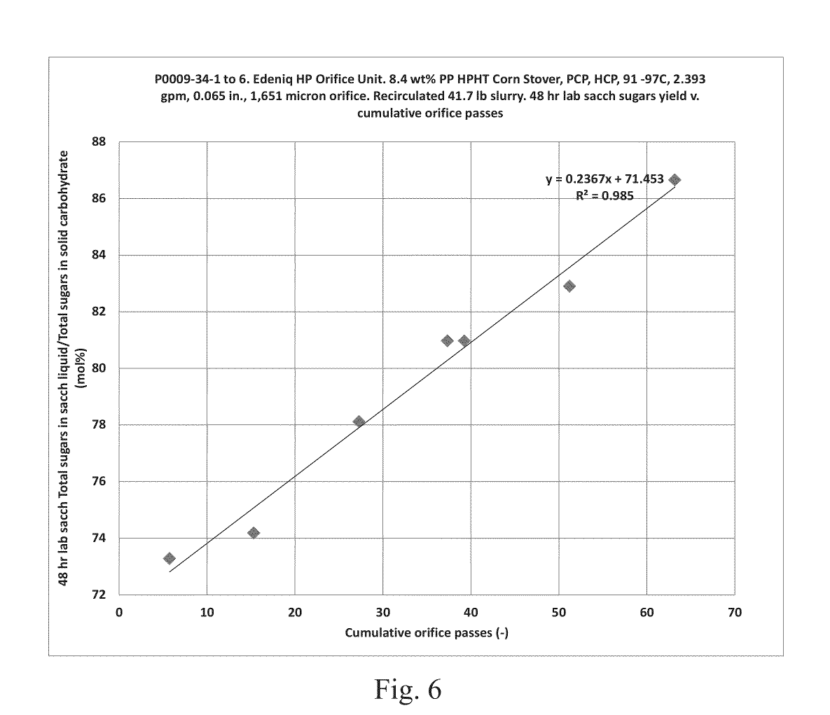

FIG. 6 applies to the same run as FIG. 5, the 48 hour saccharin sugar yields being presented here, as a function of cumulative orifice passes. Note that the ongoing orifice processing correspondingly causes more sugars to be liberated in the subsequent lab saccharification.

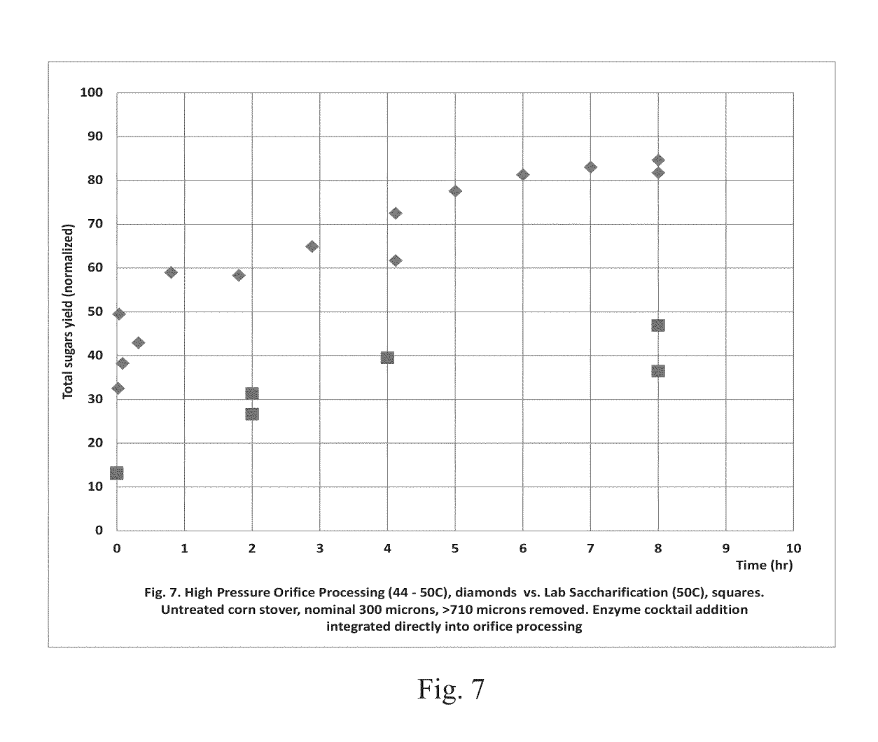

FIG. 7 compares high pressure shear (orifice) processing (diamonds) to saccharification (squares), with enzyme cocktail addition integrated directly into the shear/orifice processing. From this graph, it is evident that orifice processing with enzyme addition provides an improvement in yield of approximately 30 percentage points (i.e., from around 12% to around 40% (average) at 0 hours, from around 30% to around 60% at 2 hours, from around 40% to around 65-70% at 4 hours, and from around 40% to around 82% at 8 hours). Thus, the direct integration of enzyme introduction with high shear treatment, with high shear accomplished by the combination of pressure and orifice processing, enables substantial increase in initial saccharification rate, decrease in overall time of saccharification, and increase in ultimate sugars yield.

Conclusions:

Eight to 13 wt % pilot plant high pressure high temperature corn stover has been orifice-processed continuously without enzyme for 1 to 2+ hours under a variety of conditions.

One orifice with recirculation changes the slurry measurably within 50 passes or 1 to 2 hours.

Regarding particle size variation with orifice on stream time, the conditions either produce no apparent change, over limited processing times, at lower temperature, at lower shear rates or under more aggressive conditions of flow, orifice geometry and temperature, the particle size falls monotonically, be it the mean, median, D10 or D90.

Over the 1 to 2+ hours of the runs, linear fits of particle size v. time or orifice passes are adequate.

The highest rates of size reduction occurred at 90-95° C., rather than cooler temperatures. This is consistent with elevated temperature promoting biocellulosic particle mechanical weakness and/or greater water affinity, accelerating the process of size reduction.

The zero, 2 and 48 hour sugar releases were all promoted relative to the base case by ongoing and repeated orifice flow.

The direct integration of enzyme introduction with high shear treatment enables substantial increases in short-term saccharification rates, decrease in overall time required for saccharification, and increase in ultimate sugars yield.

Different arrangements of the components depicted in the drawings or described above, as well as components and steps not shown or described are possible. Similarly, some features and subcombinations are useful and may be employed without reference to other features and subcombinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications can be made without departing from the scope of the claims below.

We claim:

1. A method for the pretreatment of biomass solids comprising:
a. hydrating the biomass solids to form a biomass slurry;
b. shear treating the biomass solids to reduce the particle size of the biomass solids, modify particle or slurry morphology, or rupture the cell walls of the biomass solids; and
c. hydrolyzing the biomass solids in the presence of reactive enzymes in a pressure hydrolysis zone,
wherein the pressure hydrolysis zone comprises a high-shear, high-pressure, low-temperature heat exchange and reaction zone and a low-pressure, low-temperature polishing zone.

2. The method of claim 1, wherein the heat exchange and reaction zone comprises a plug flow reactor that provides for radial mixing and intentionally limited back mixing of the biomass solids in the biomass slurry to provide sustained contact between the biomass solids and the reactive enzymes and facilitate conversion of the biomass solids into sugar-rich intermediates.

3. The method of claim 2, wherein the polishing zone comprises a continuous stirred tank reactor that provides additional residence time to further facilitate conversion of the biomass solids into sugar-rich intermediates.

4. The method of claim 2, wherein the plug flow reactor operates at a pressure of from about 1,000 psi to about 10,000 psi and a temperature of from about 25° C. to about 140° C.

5. The method of claim 2, wherein the plug flow reactor operates at a pressure of from about 1,000 psi to about 5,000 psi and a temperature of from about 35° C. to about 100° C.

6. The method of claim 2, wherein the plug flow reactor operates at a pressure of from about 1,000 psi to about 2,500 psi and a temperature of from about 35° C. to about 50° C.

7. The method of claim 3, wherein the continuous stirred tank reactor operates at an operating temperature of less than about 70° C. and at a pressure corresponding to the saturation pressure of the biomass slurry at the operating temperature.

8. The method of claim 1, wherein the step of hydrating the biomass solids comprises a continuous process comprising adding coarsely ground biomass solids into a water stream in a disperser to form the biomass slurry and then passing the biomass slurry into a heat exchanger.

9. The method of claim 8, wherein the operating temperature of the heat exchanger is from about 120° C. to about 250° C. and the pressure of the heat exchanger corresponds to the saturation pressure of the biomass slurry at the operating temperature.

10. The method of claim 8, wherein the operating temperature of the heat exchanger is about 180° C. and the pressure of the heat exchanger corresponds to the saturation pressure of the biomass slurry at the operating temperature.

11. The method of claim 8, further comprising adding doping chemicals to the biomass slurry for pH adjustment or control.

12. The method of claim 8, further comprising adding one or more enzymes to the biomass slurry to help initiate or accelerate downstream enzymatic hydrolysis reactions.

13. The method of claim 8, wherein the biomass slurry comprises at least about 13 weight percent solids.

14. The method of claim 8, wherein the biomass slurry comprises at least about 20 weight percent solids.

15. The method of claim 8, wherein the biomass slurry comprises at least about 30 weight percent solids.

16. The method of claim 1, wherein the step of shear treating the biomass solids comprises passing the biomass slurry through at least two particle size reduction mills arranged in a series configuration.

17. The method of claim 16, wherein the at least two particle size reduction mills reduce a substantial amount of the biomass solids to a particle size of less than about 100 microns.

18. The method of claim 16, wherein the at least two particle size reduction mills reduce a substantial amount of the biomass solids to a particle size of less than about 50 microns.

19. The method of claim 16, wherein the at least two particle size reduction mills reduce a substantial amount of the biomass solids to a particle size of less than about 30 microns.

20. The method of claim 1, wherein the modification of particle morphology arises from frictional, impact, centrifugal or cavitational forces and results in the cellular liberation of saccharides or saccharide precursors.

21. The method of claim 16, wherein the operating temperature of the biomass slurry in the particle size reduction mills is from about 120° C. to about 250° C., and the pressure of the biomass slurry corresponds to the saturation pressure of the biomass slurry at the operating temperature.

22. A method for the pretreatment of biomass solids comprising:
a. hydrating the biomass solids to form a biomass slurry in a continuous process comprising adding coarsely ground biomass solids into a water stream in a disperser to form the biomass slurry and then passing the biomass slurry into a heat exchanger;
b. shear treating the biomass solids by passing the biomass slurry through at least two particle size reduction mills arranged in a series configuration to reduce the particle size of the biomass solids or rupture the cell walls of the biomass solids; and
c. hydrolyzing the biomass solids in the presence of reactive enzymes in a pressure hydrolysis zone,
wherein the pressure hydrolysis zone comprises a high-shear, high-pressure, low-temperature heat exchange and reaction zone and a low-pressure, low-temperature polishing zone.

23. The method of claim 22, wherein the method results in a sugar-rich aqueous solution suitable for subsequent chemical, biochemical or enzymatic conversion to valuable fuels, chemicals, or solvents.

24. The method of claim 23, wherein the sugar-rich aqueous solution is suitable for synthesis of gasoline-like, jet fuel-like, or diesel-like surrogates, additives, or alternatives.
